(19) United States
(12) Patent Application Publication
(10) Pub. No.: US 2024/0058397 A1
(43) Pub. Date: Feb. 22, 2024

(54) Probiotic Formulation for vaginal health

(71) Applicant: RATNA SUDHA MADEMPUDI, Hyderabad (IN)

(72) Inventors: RATNA SUDHA MADEMPUDI, Hyderabad (IN); Jayesh Ahire, Hyderabad (IN); G Suvarna Iakshmi, Hyderabad (IN); Y Mahalaxmi, Hyderabad (IN); Suhasini Sahoo, Hyderabad (IN); Megha Kashikar, Hyderabad (IN); Ambika Heerekar, Hyderabad (IN)

(21) Appl. No.: 18/235,910

(22) Filed: Aug. 21, 2023

(30) Foreign Application Priority Data
Aug. 20, 2022 (IN) 202241047456

(51) Int. Cl. A61K 35/747; A61K 47/26; A61K 31/375; A61K 9/00; A61P 31/10; A61P 31/04

(52) U.S. Cl. CPC A61K 35/747; A61K 47/26; A61K 31/375; A61K 9/0034; A61P 31/10; A61P 31/04

(57) ABSTRACT

The current invention discloses a probiotic formulation comprising a prebiotic and the Lactobacilli strains Lactobacillus crispatus UBLcP-01, Lactobacillus gasseri UBLG-36, and Lactobacillus johnsonii UBLJ-01 strains.

The current invention also encompasses methods for treating, ameliorating and/or preventing vaginitis caused by infections and inflammatory conditions, using the probiotic formulation described herein.

### REFERENCES

1. Ahire. J. J., Sahoo. S., Kashikar, M. S., Heerekar, A., Lakshmi, S. G., & Madempudi, R. S. (2023) In Vitro Assessment of Lactobacillus crispatus UBLCp01, Lactobacillus gasseri UBLG36, and Lactobacillus johnsonii UBLJ01 as a Potential Vaginal Probiotic Candidate. Probiotics and antimicrobial proteins, 15(2), 275-286.
2. Giordani. B., Naldi, M., Croatti, V., Parolin, C., Erdoǵan, Ü., Barolini, M., & Vitali, B. (2023). Exopolysaccharides from vaginal lactobacilli modulate microbial biofilms. Microbial cell factories. 22(1), 45.
3. Kaewsrichan, J., Peevananjarassri, K., & Kongprasertkit, J. (2006). Selection and identification of anaerobic lactobacilli producing inhibitory compounds against vaginal pathogens. FEMS immunology and medical microbiology, 48(1), 75-83
4. Paladme, H. L., & Desai, U. A. (2018). Vaginitis: Diagnosis and Treatment. American family physician, 97(5), 321-329.
5. Wilson W W, Wade M M, Holman S C, Champlin F R. (2001). Status of methods for assessing bacterial cell surface charge properties based on zeta potential measurements. J. Microbiol. Methods 43: 153-164.

### FIELD OF INVENTION

The current invention relates to the field of vaginal probiotics. The current invention more specifically relates to methods and compositions comprising novel strains of Lactobacillus that can be used for enhancing vaginal health.

### BACKGROUND

Infections and inflammatory conditions of the urogenital tract, are frequently experienced by women all over the world, and these conditions are exacerbated by the changes in the reproductive tract during a woman's life cycle. One example of such a condition is vaginitis, which is an inflammation of the vulvovaginal region.

Vaginitis includes the conditions associated with a perturbation in the composition of the normal vaginal microflora.

The composition of the vaginal microbiota changes during the lifetime of woman, i.e., from birth, puberty, reproductive age, and pre-menopause to menopause. Also, vaginal health is dependent to a great extent on the presence of Lactobacilli species. Perturbation in the composition of vaginal microbiota may involve depletion of Lactobacilli species and overgrowth of other bacterial species, such as Gardnerella vaginalis and obligate anaerobes, resulting in inflammation and infection. Overall, dysbiosis of vaginal microbiota results in the development of gynecologic and obstetric diseases. Bacterial vaginosis (BV) is a prevalent disorder affecting the lower genital tract in reproductive-age women. It is characterized by a decrease in the number of beneficial Lactobacilli and increase in facultative or obligate anaerobic microbes (Atopobium, Bifidobacterium, Gardnerella, Leptotrichia, Mobiluncus, Prevotella, Sneathia, and members of Clostridiales). BV prevalence is associated with sexually transmitted diseases, miscarriage, preterm birth, low birth weight, and pelvic inflammatory diseases. Worldwide, 23 to 29% of women get infected with BV annually and 84% of these affected women do not exhibit symptoms. Amoxycillin, clindamycin, and metronidazole are usually employed to treat BV; however, reinfections are common. Moreover, the antibiotic treatment kills beneficial vaginal bacteria disturbing the microbiome composition, and may lead to the development of antibiotic resistance and other fungal diseases. Candidal vaginitis is typically caused by overgrowth of Candida yeast, it is most commonly overgrowth of the commensal fungal organism Candida albicans.

Vaginitis symptoms may include change in colour, odour or amount of discharge from a subject's vagina, vaginal itching or irritation, pain during intercourse, painful urination, and light vaginal bleeding or spotting. Vaginitis can lead to various degrees of discomfort and can lower the overall quality of life. Vaginitis can also have serious medical consequences. For example, BV may increase the risk of HIV acquisition, premature labor in pregnant subjects and low birth weight in their babies. Vaginitis therefore needs to be promptly treated to improve the quality of life and to avoid medical complications. Urogenital infections are typically treated with various antimicrobial treatments, including antibiotics and antifungal agents, but the recurrence rate is high and side effects are common. Improved compositions and methods for treatment, alleviation or prevention of vaginal infections and inflammation are desirable.

The use of beneficial bacteria has recently gained a lot of interest in preventing and treating diseases such as vaginitis that are cause by imbalance of types of bacterial population or microbiome. Considering the dominance of several types lactobacilli species and strains in the vagina of healthy women, there is a lot of scope to search and evaluate strains for beneficial characteristics to improve vaginal health.

The current invention discloses a probiotic formulation for enhancing vaginal health, comprising the novel strains Lactobacillus crispatus UBLcP-01, Lactobacillus gasseri UBLG-36, and Lactobacillus johnsonii UBLJ-01 isolated from the vagina of healthy individuals.

These novel strains show low pH tolerance, survival in simulated vaginal fluid, adhesion abilities, safety, and antimicrobial potential, exopolysaccharide production, and biofilm-forming abilities, that make these strains good probiotic candidates for promoting vaginal health.

### SUMMARY

The current invention discloses compositions and methods for treatment and/or management of vaginitis in women.

The current invention encompasses a composition for treating vaginitis comprising dried live bacteria of the strains Lactobacillus crispatus UBLcP-01, Lactobacillus gasseri UBLG-36, Lactobacillus johnsonii UBLJ-01 and a prebiotic.

In one embodiment, the composition comprises a prebiotic which is selected from the group consisting of inulin, Fructooligosaccharides (FOS), Galacto-oligosaccharides (GOS), and lactulose. In one embodiment, the prebiotic is present in the composition at a concentration 25-30% w/w.

In one embodiment, the composition disclosed herein further comprises an antioxidant selected from the group consisting of ascorbic acid, vitamin E, glutathione, coenzyme Q10, beta-carotene, lycopene, vitamin A or derivatives thereof and the antioxidant is present at a concentration of 8-12% w/w in the composition.

In one embodiment, the antioxidant is ascorbic acid or a salt thereof. In one embodiment, the ascorbic acid or its salt thereof is present at a concentration of 10% (w/w) in the composition.

In one embodiment, the composition disclosed herein comprises 0.1-5 billion cfu of Lactobacillus crispatus UBLcP-01, 0.1-5 billion cfu of Lactobacillus gasseri UBLG-36, and 0.1-5 billion cfu of Lactobacillus johnsonii UBLJ-01. In one embodiment, the composition disclosed herein comprises 0.1-1 billion cfu of Lactobacillus crispatus UBLcP-01, 0.1-5 billion cfu of Lactobacillus gasseri UBLG-36, and 0.1-5 billion cfu of Lactobacillus johnsonii UBLJ-01. In one embodiment, the composition comprises total 1-3 billion cfu of Lactobacillus crispatus UBLcP-01, Lactobacillus gasseri UBLG-36, and Lactobacillus johnsonii UBLJ-01. In one embodiment, the composition comprises total 1 billion cfu of Lactobacillus crispatus UBLcP-01, Lactobacillus gasseri UBLG-36, and Lactobacillus johnsonii UBLJ-01.

In one embodiment, the composition disclosed herein further comprises an antifungal agent.

In one embodiment, the composition is in the form of a freeze-dried preparation, cream, paste, gel liquid, suppository, capsule, or tablet for vaginal application.

In one embodiment, the composition disclosed herein is in form of vaginal suppository.

One embodiment of the current invention is a method of treating or preventing vaginitis in female subjects, the method comprising the steps of:
(a) providing a composition comprising the following ingredients per single dosage:
   (i) a prebiotic; and (ii) dried live bacteria of the strains Lactobacillus crispatus UBLcP-01, Lactobacillus gasseri UBLG-36, and Lactobacillus johnsonii UBLJ-01; and
(b) administering the composition intravaginally to the subject in need of treatment.

In one embodiment, the method disclosed herein, wherein the prebiotic is selected from the group consisting of inulin, Fructooligosaccharides (FOS), Galacto-oligosaccharides (GOS), and lactulose. In one embodiment, the prebiotic is present in the composition at a concentration 25-30% w/w.

In one embodiment, the method disclosed herein, wherein the composition further comprises an antioxidant selected from the group consisting of ascorbic acid, vitamin E, glutathione, coenzyme Q10, beta-carotene, lycopene, vitamin A or derivatives thereof and the antioxidant is present at a concentration of 8-12% w/w in the composition. In one embodiment, the antioxidant is ascorbic acid or a salt thereof. In one embodiment, the ascorbic acid or its salt thereof is present at a concentration of 10% (w/w) in the composition.

In one embodiment, the method disclosed herein,
the composition disclosed herein comprises 0.1-5 billion cfu of Lactobacillus crispatus UBLcP-01, 0.1-5 billion cfu of Lactobacillus gasseri UBLG-36, and 0.1-5 billion cfu of Lactobacillus johnsonii UBLJ-01. In one embodiment, the composition disclosed herein comprises 0.1-1 billion cfu of Lactobacillus crispatus UBLcP-01, 0.1-5 billion cfu of Lactobacillus gasseri UBLG-36, and 0.1-5 billion cfu of Lactobacillus johnsonii UBLJ-01.

In one embodiment, the composition comprises total 1-3 billion cfu of Lactobacillus crispatus UBLcP-01, Lactobacillus gasseri UBLG-36, and Lactobacillus johnsonii UBLJ-01. In one embodiment, the composition comprises total 1 billion cfu of Lactobacillus crispatus UBLcP-01, Lactobacillus gasseri UBLG-36, and Lactobacillus johnsonii UBLJ-01.

In one embodiment, the method disclosed herein, wherein the composition is in the form of a freeze-dried preparation, cream, paste, gel liquid, suppository, capsule, or tablet for vaginal application.

In one embodiment, the composition is administered to the subject once or twice a day.

### BRIEF DESCRIPTION OF FIGURES

FIG. 1 shows bacterial growth of the strains Lactobacillus crispatus UBLcP-01, Lactobacillus gasseri UBLG-36, and Lactobacillus johnsonii UBLJ-01 in low pH (6.5, 4.5, 4.0, and 3.5) media (acidified with lactic acid) at 0, 24, and 48 h of anaerobic incubation. All data are represented as mean±SD. a, p≤0.0001: b, p≤0.001; d, p≤0.05; and e, p>0.05.

FIG. 2 shows bacterial growth of the three strains Lactobacillus crispatus UBLcP-01, Lactobacillus gasseri UBLG-36, and Lactobacillus johnsonii UBLJ-01 in simulated vaginal fluid (SVF) and MRS broth at 0, 24, and 48 h of anaerobic incubation. FIG. 2a) shows changes in viability and FIG. 2b shows the pH units. All data are represented as mean±SD. a, p≤0.0001; b, p≤0.001; c, p≤0.01; d, p≤0.05; and e, p>0.05.

FIG. 3 shows quantification of adhesion of the three strains Lactobacillus crispatus UBLcP-01, Lactobacillus gasseri UBLG-36, and Lactobacillus johnsonii UBLJ-01 to mucin by crystal violet (CV) staining. All data are represented as mean±SD. ****p≤0.0001.

FIG. 4 shows antimicrobial activity of bacterial strains against selected pathogens. Data points presented are the average of 3 independent experiments. ****p≤0.0001, ***p≤0.001.

FIG. 5 shows biofilm formation ability of the three bacterial strains Lactobacillus crispatus UBLcP-01, Lactobacillus gasseri UBLG-36, and Lactobacillus johnsonii UBLJ-01 at 0 and 24 h of anaerobic incubation. FIG. 5a shows total biofilm formation by crystal violet method. FIG. 5b shows Viable cells in total biofilm. c Planktonic cells. ****p≤0.0001, ns: p>0.05.

FIG. 6 shows adhesion studies of individual cells and Blends. The figure shows Vk2 cells treated with (A) L. crispatus UBLcP-01 (B) L. gasseri UBLG-36 (C) L. johnsoni UBLJ-01 (D) Blend1. (E) Blend 2, (F) Blend 3 (G) control (without any bacterial cells).

FIG. 7 shows cytokine profile in vitro with and without Gardnerella vaginalis (GV) in presence of blend 1, blend 2 and blend 3 of the three strains Lactobacillus crispatus UBLcP-01, Lactobacillus gasseri UBLG-36, and Lactobacillus johnsonii UBLJ-01.

### DETAILED DESCRIPTION

The composition of the vaginal microbiota changes during the lifetime of woman, from birth, puberty, reproductive age, and pre-menopause to menopause. Moreover, vagina health is dependent on the presence of Lactobacillus species. Thus, dysbiosis of vaginal microbiota results in the development of gynecologic and obstetric diseases.

Multiple research studies reveal that beneficial microbes are an essential part of the microbiota ecosystem in humans and they outnumber human cells approximately by 10-fold. Imbalance of these microbial population often leads to alteration of the host immunity and sometimes even host genetics and may impact the integrity of the host system, increase the colonization of pathogenic bacteria, and also modulate the production of neurotransmitters, proinflammatory cytokines, neurotrophic factors, hormones, neuropeptides and amino acid, and carbohydrate metabolisms.

Vaginal fluid is a mixture of secretions from Bartholin- and Skenes's-glands, tubal- and endometrial-fluids, and cervical mucus. It is a complex fluid which contains ˜90-95% water, mucins, carbohydrates, salts (organic and inorganic), urea, fatty acids, albumin, and other macromolecules.

Naturally occurring bacteria in the vagina help to avoid the growth and proliferation of pathogens. Lactobacilli which are the dominant bacteria in the vaginal flora, maintain an environment that restricts the growth of pathogenic microorganisms using properties such as steric exclusion and inhibitory substance production. Lactic acid produced from the metabolism of carbohydrates is one such inhibitory substance, which helps to maintain the vaginal pH at <4.5, thereby creating an inhospitable environment for the growth of most endogenous pathogenic bacteria. Hydrogen peroxide (H₂O₂) is usually generated by lactobacilli present in a healthy vagina, but is mostly absent in women with bacterial vaginosis. H₂O₂-dependent activity of bacteria beneficial to vaginal health, is bactericidal to Gardnerella vaginalis in vitro.

A deficiency in Lactobacilli can upset the microbial balance in the vagina, resulting in the bacterial vaginosis (BV), which may be associated with a quantitative and qualitative shift from normally occurring Lactobacilli to a mixed flora dominated by anaerobic bacteria. Bacterial vaginosis may be characterized by a complete loss of Lactobacilli and a concomitant increase in Gram-variable and Gram-negative rods, primary among them Gardnerella vaginalis as well as Bacteroides, Prevotella, and Mobiluncus species. Loss of vaginal Lactobacilli also leaves nonpregnant women susceptible to infection which may result in endometritis or even pelvic inflammatory disease.

BV prevalence is associated with sexually transmitted diseases, miscarriage, preterm birth, low birth weight, and pelvic inflammatory diseases. Worldwide, annually 23 to 29% of women get infected with BV and 84% reported no symptoms (Ref 1: Ahire et al). Amoxycillin, clindamycin, and metronidazole are usually employed to treat BV: however, reinfections are common. Besides this, the antibiotic treatment kills beneficial vaginal bacteria disturbing the microbiome composition, and may lead to the development of antibiotic resistance and other fungal diseases.

During menopause, several changes occur in the female genital tract. The major universal change is vaginal atrophy. Vaginal dryness, burning, itching and dyspareunia are frequent complaints along with dysuria, urinary frequency and recurrent infections. The genitourinary atrophy following menopause is associated with a decline in estrogen secretion accompanied by depletion of lactobacilli and increased colonization by pathogenic microorganisms associated with bacterial vaginosis and urinary tract infections. In post-menopausal women, vaginal estriol therapy reduces E. coli colonization and increases the numbers of Lactobacilli, with the result that the incidence of recurrent urinary and genital tract infections drops significantly. All these indications combined with BV lead to vaginitis in women, which can lead to serious health implications.

Most of the probiotic strains that have been isolated from vaginal samples from healthy women till now, are unable to survive in an acidic pH environment, or are unable to persist/colonize as biofilms in the vagina, or to survive extended contact with bile. Vaginal pH varies significantly during a woman's life: physiological pH is 7 in prepubescent girls, drops to 3.8 in pubescent females, and is greater than 5.5 during menopause, in the absence of hormone replacement therapy. These variations are related to multiple factors such as the presence of estrogen and colonization by lactobacilli.

The current invention discloses a composition comprising a prebiotic, three lactobacilli strains that show beneficial properties. The composition disclosed herein and comprising a prebiotic and Lactobacillus crispatus UBLcP-01, Lactobacillus gasseri UBLG-36, and Lactobacillus johnsonii UBLJ-01 isolated from the vagina of healthy reproductive age Indian women showed the ability to survive acidic and simulated vaginal fluid conditions and could adhere to mucin. Lact. gasseri UBLG-36, and Lact. johnsonii UBLJ-01 produce D- and L-lactic acid, whereas Lact. crispatus UBLcP-01 produces hydrogen peroxide and D- and L-lactic acid. The composition disclosed herein inhibits the growth of pathogens (Escherichia coli, Gardnerella vaginalis, Proteus mirabilis, and Candida albicans) and is capable of co-aggregating with them with varying degrees, and forms biofilms.

### Definitions

As used herein, the term “microbiota” is used to refer to the compilation/collection of bacterial microorganisms within a specific environment.

As used herein, the term “microbiome” refers to the bacterial taxa in a specific environment, and also their collective genomes.

The term “probiotic” as used herein is defined as a live microorganism which when administered in adequate amounts confers a health benefit on the host.

As used herein, the term “probiotic” encompasses bacterial cells beneficial to the host. Probiotics are mainly characterized by lack of pathogenicity, tolerance to gastrointestinal conditions (acid and bile), ability to adhere to the gastrointestinal mucosa and competitive or active exclusion of pathogens. These and other favorable probiotic features for defending against specific pathogens are strain-dependent, even among bacteria of the same species. Therefore, it is important to find those strains that have a better performance in all probiotic requirements. Kaewsrichan et al (Ref 3) describes selection of three Lactobacillus crispatus strains with properties relevant to mucosal colonization and the production of H₂O₂ and/or bacteriocin-like compound, from a group of 100 tested strains isolated from the vaginas of healthy premenopausal women. Hence specific strain selection is highly important for obtaining desirable properties and excluding undesirable properties for any probiotic formulation development. (Ref 3: Kaewsrichan et al; Ref 2: Giordani et al). Moreover, probiotic formulation with more than one strain also often gives unforeseen advantages such as increased ability to form aggregates, or co-aggregates, or increased adherence to epithelial cells.

The term “vaginitis” as used herein refers to any condition with symptoms of abnormal vaginal discharge, odor, irritation, itching, or burning. The most common causes of vaginitis are bacterial vaginosis, vulvovaginal candidiasis, and trichomoniasis (Ref 4: Paladine, H. L., & Desai, U. A.).

Bacterial vaginosis refers to an imbalance of the vagina's microbial flora. It is characterized by the disappearance of lactobacilli and the multiplication of pathogenic bacteria such as those listed above.

The term “candidiasis” refers to a vaginal infection caused by yeasts of the genus Candida. Bacterial vaginosis is implicated in 40% to 50% of vaginitis cases with identified cause, with vulvovaginal candidiasis accounting for 20% to 25% and trichomoniasis for 15% to 20% of cases. Other causes of vaginitis are non-infectious causes, including atrophic, irritant, allergic, and inflammatory vaginitis. Diagnosis of the causation of vaginitis is made by studying the combination of symptoms, physical examination findings, and office-based or laboratory testing. Bacterial vaginosis is traditionally diagnosed with Amsel criteria, although Gram stain is the diagnostic standard. Newer laboratory tests that detect Gardnerella vaginalis DNA or vaginal fluid sialidase activity have similar sensitivity and specificity to Gram stain. Bacterial vaginosis is treated with oral metronidazole, intravaginal metronidazole, or intravaginal clindamycin. The diagnosis of vulvovaginal candidiasis is made using a combination of clinical signs and symptoms with potassium hydroxide microscopy: DNA probe testing is also available. Culture can be helpful for the diagnosis of complicated vulvovaginal candidiasis by identifying nonalbicans strains of Candida. Treatment of vulvovaginal candidiasis involves oral fluconazole or topical azoles, although only topical azoles are recommended during pregnancy. Nucleic acid testing might be done for the diagnosis of trichomoniasis in symptomatic or high-risk women. Trichomoniasis is treated with oral metronidazole or tinidazole, and patients' sex partners should be treated as well. Standard treatment of non-infectious vaginitis is directed at the underlying cause. Atrophic vaginitis is treated with hormonal and nonhormonal therapies. Standard treatment of inflammatory vaginitis is topical clindamycin as well as steroid application.

As used herein, the term “prebiotic” refers to a nutritional supplement for the probiotic bacterium. Prebiotics can be food ingredients, for example, oligosaccharides, that are non-digestible by a subject (e.g., by a mammal such as a human), and that stimulates the growth or activity of one or more beneficial bacteria and/or inhibit the growth or activity of one or more pathogenic bacteria. A prebiotic may selectively stimulate the growth and/or activity of one or a limited number of bacteria in the subject.

Examples of prebiotics include, but are not limited to inulin, Fructooligosaccharides (FOS), Galacto-oligosaccharides (GOS), lactulose, extracts of artichoke, chicory root, oats, barley, various legumes, garlic, kale, beans or flacks or an herb.

As used herein, the term antioxidant refers to a molecule that helps to protect the composition during drying, storage and/or reconstitution, by its anti-oxidation effects.

As used herein, the term “autoaggregation” is defined as the ability of the probiotic bacterial cells to clump with cells of the same species.

Autoaggregation correlates with adhesion of bacteria to epithelial cells, which is a prerequisite for colonization and infection of the mucosal linings by many pathogens.

As used herein, the term “coaggregation” or “co-aggregation” is defined as the ability of the probiotic bacteria to interact closely and clump/aggregate with other bacteria such as pathogens.

In order to manifest beneficial effects, probiotic bacteria need to achieve an adequate mass through aggregation. Consequently, the ability of probiotics to aggregate is a desirable property. Organisms with the ability to coaggregate with other bacteria such as pathogens also has an advantage over non-coaggregating organisms, which are more easily removed from the intestinal environment.

As used herein, the term “adherence” refers to adhering of bacteria to epithelial cells, which might be vaginal cells in context of the current invention. Adherence of bacteria to intestinal epithelium is known to be a prerequisite for colonization. Adhesion is a complex process involving non-specific (hydrophobicity) and specific ligand-receptor mechanisms. Adherence of bacterial cells is usually related to cell surface characteristics.

As used herein, the term “Zeta potential” is defined as the difference in electrical potential between the surface of the bacterium and the bulk surrounding medium. It is a measure of the net distribution of electrical charge on the surface of the bacterium. Hence, zeta potential of a bacterial cell, which is a measure of the electrical surface charge of a bacterial particle in a suspension is determined by the nature of the groups exposed at the surface, and under normal physiological conditions bacteria are usually negatively charged due to the large amount of phosphate and carboxyl groups present on the cell surface. Zeta potential measurement is often used to characterize the surface properties of intestinal bacteria and as an indicator of their viability, integrity, efficacy and adhesion potential to epithelial cells (Ref 5: Wilson et al).

Exopolysaccharides (EPS) are high molecular-weight biopolymers produced and often secreted by several microorganisms. They consist of repeating units of one (homopolysaccharides) or more (heteropolysaccharides) monosaccharides with (or without) non-carbohydrate substituents. EPS are tightly attached to bacterial surfaces, loosely associated with the cell envelope, or secreted into the surrounding environment. Bacterial EPS playa role in one or more of several activities such as cell recognition, bacterial adhesion and communication, as protective agents and many times as components of biofilm extracellular matrix. Studies have shown that EPS play a role in biofilm formation (Ref 2: Giordani et al).

As used herein, the term “biofilm” refers to organized communities of microorganisms, often of different species, enclosed in a self-produced polymer matrix and attached to an inorganic or organic surface. The biofilms are very resistant to antimicrobial agents.

In the vaginal environment, the naturally occurring beneficial lactobacilli can organize themselves into physiological biofilms which are one of the indications of a healthy vaginal ecosystem.

One of the factors that limits the beneficial effects of the probiotic strains used currently for treating vaginal infections is the inability of these strains to establish biofilms even under optimal conditions, minimizing the magnitude and duration of their healthful effects.

As used herein, the terms “composition” and “formulation” are used interchangeably herein.

As used herein, “treating and/or preventing vaginitis” includes, the amelioration or alleviation, at least in part, of one or more symptoms of vaginitis. For example, symptoms of vaginitis, and related disorders that may be inhibited, or alleviated include decrease in inflammation, reduction or killing of pathogenic microbes inhabiting the vagina, maintenance of desired vaginal pH, decrease in recurrence of bacterial or fungal infections of the vagina. “Treating or preventing” also include preventing the onset of symptoms in a subject, or preventing the appearance of symptoms.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the “prevention/preventing”, “alleviation/alleviating”, “control/controlling”, “reduction/reducing”, “management/managing” or “mitigation/mitigating” means preventing the occurrence of symptoms that may be associated with vaginitis.

A “subject” or “patient” is a person who may be predisposed vaginitis or may display one or more symptoms of such a condition.

As used herein, the term “effective amount” or “therapeutically effective amount” refers to the amount of a compound sufficient to effect beneficial or desired results. An effective amount can be administered in one or more administrations, applications or dosages and is not intended to be limited to a particular formulation or administration route. It includes an amount of a compound of the invention that is effective when administered alone or in combination to treat or prevent the onset of the disease condition or disorder. For a combination of compounds, each with a therapeutic effect, “therapeutically effective amount” of the combination of compounds is an amount of the combination of compounds claimed that is effective to treat the disease condition or disorder. The combination of compounds can be additive and is preferably a synergistic combination.

Inflammation is a complex process with a variety of cell and signalling proteins that protect the body. At times, the immune system may trigger an inflammatory response inappropriately, causing autoimmune diseases. When the inflammation process starts, chemicals in white blood cells are released into the blood and the affected tissues to protect the body. Five cardinal signs of inflammation are pain, heat, redness, swelling, which can in many instances, lead to loss of function.

There are two types of inflammation, acute and chronic. Studies show that the symbiotic relationship between the human microbiota and the overall functioning of the body is often disturbed in a variety of both acute and chronic diseases/disorders.

Treatment for inflammatory responses usually depends on the specific disease or ailment and the severity of symptoms. However, the standard care of treatment for acute inflammation includes:

Bacterial infection: Antibiotics such as Doxycycline, Clindamycin, Clofazimine, Metronidazole, etc.

Nonsteroidal anti-inflammatory drugs (NSAIDs): NSAIDs are the first-line treatment for short-term pain and inflammation in general. NSAIDs such as aspirin, ibuprofen, and naproxen are the usually consumed drugs for inflammation Based on the severity of acute inflammatory conditions and patient history, prescription-strength NSAIDs may also be prescribed.

Corticosteroids: These are steroids commonly available in pill form and as injections, that are used to treat swelling and inflammation for short periods, since they are known to cause serious side effects.

Topical medications: Topical medications may also include analgesics and steroids, which can help with both acute and chronic pain and inflammation of the skin and joints, without the side effects of oral treatments. These are helpful for managing long-term diseases/disorders of inflammation and contain an NSAID, such as diclofenac or ibuprofen.

Cytokines:

Cytokines are a broad group of signalling proteins released into the bloodstream which cause vascular permeability, or the ability of molecules to pass through blood vessels. They modulate the functions of individual cells, and regulate processes taking place under normal, developmental and pathological conditions. The major cytokine receptor families include, type I cytokine receptors, type 11 cytokine receptors, TNF receptors, IL-1 receptors, tyrosine kinase receptors, and chemokine receptors.

Cytokines are redundant in their activity, meaning similar functions can be stimulated by different cytokines and can act synergistically or antagonistically. They are frequently produced in a cascade, stimulating its target cells to make additional cytokines. Proinflammatory cytokines are involved in the development of inflammatory and neuropathic pain. They are produced mainly by activated macrophages and are involved in the up-regulation of inflammatory reactions. These include IL-1β, IL-6, and TNF-α involved in the process of pathological pain.

The anti-inflammatory cytokines, on the other hand are a series of immunoregulatory molecules that control the pro-inflammatory cytokine response. Major anti-inflammatory cytokines include interleukin (IL)-1 receptor antagonist, IL-4, IL-10, IL-11, and IL-13. Leukaemia inhibitory factor, interferon-alpha, IL-6, and transforming growth factor (TGF)-β are categorized as either anti-inflammatory or pro-inflammatory cytokines, under various circumstances. Among all the anti-inflammatory cytokines, IL-10 is a cytokine with potent anti-inflammatory properties, repressing the expression of inflammatory cytokines such as TNF-α, IL-6 and IL-1 by activated macrophages.

The current invention discloses a composition comprising a prebiotic and three novel strains of Lactobacillus, isolated from the vaginal tract of healthy Indian women. The strains Lactobacillus crispatus UBLcP-01, Lactobacillus gasseri UBLG-36, and Lactobacillus johnsonii UBLJ-01 isolated from the vagina of healthy reproductive age Indian women disclosed herein exhibit beneficial probiotic properties.

All the three strains have been deposited in the IDAs affording permanence of the deposit and ready accessibility thereto by the public if a patent is granted.

Lactobacillus crispatus UBLcP-01 was deposited in MTCC. The Microbial Type Culture Collection and Gene Bank (MTCC facility in IMTECH, Sector-39, Chandigarh, India-160036) (https://mtccindia.res.in/), is an affiliate member of the World Federation for Culture Collections (WFCC) and is registered with the World Data Centre for Microorganisms (WDCM).

Lactobacillus gasseri UBLG-36 and Lactobacillus johnsonii UBLJ-01 were deposited in the IDA Microbial culture collection (MCC: National Centre for Cell Science (NCCS), University of Pune Campus, Ganeshkhind, Pune-411007, Maharashtra, India).

TABLE 1

| Strain | IDA | Strain Number |
|---|---|---|
| Lactobacillus crispatus UBLcP-01 | MTCC | MTCC 25603 |
| Lactobacillus gasseri UBLG-36 | MCC | MCC 0139 |
| Lactobacillus johnsonii UBLJ-01 | MCC | MCC 0145 |

### Embodiments

The current invention encompasses a composition for treating vaginitis comprising at least one prebiotic and dried live bacteria of the strains Lactobacillus crispatus UBLcP-01, Lactobacillus gasseri UBLG-36, Lactobacillus johnsonii UBLJ-01.

In one embodiment, the prebiotic is selected from the group consisting of inulin, Fructooligosaccharides (FOS), Galacto-oligosaccharides (GOS), and lactulose. In one embodiment, the prebiotic is present in the composition at a concentration 25-30% w/w.

In one embodiment, the composition disclosed herein further comprises an antioxidant selected from the group consisting of ascorbic acid, vitamin E, glutathione, coenzyme Q10, beta-carotene, lycopene, vitamin A or derivatives thereof and the antioxidant is present at a concentration of 8-12% w/w in the composition.

In one embodiment, the antioxidant is ascorbic acid or a salt thereof. In one embodiment, the ascorbic acid or its salt thereof is present at a concentration of 10% (w/w) in the composition.

In one embodiment, the composition disclosed herein comprises 0.1-5 billion cfu of Lactobacillus crispatus UBLcP-01, 0.1-5 billion cfu of Lactobacillus gasseri UBLG-36, and 0.1-5 billion cfu of Lactobacillus johnsonii UBLJ-01. In one embodiment, the composition disclosed herein comprises 0.1-1 billion cfu of Lactobacillus crispatus UBLcP-01, 0.1-1 billion cfu of Lactobacillus gasseri UBLG-36, and 0.1-1 billion cfu of Lactobacillus johnsonii UBLJ-01. In one embodiment, the composition comprises total 1-3 billion cfu of Lactobacillus crispatus UBLcP-01, Lactobacillus gasseri UBLG-36, and Lactobacillus johnsonii UBLJ-01. In one embodiment, the composition comprises total 1 billion cfu of Lactobacillus crispatus UBLcP-01, Lactobacillus gasseri UBLG-36, and Lactobacillus johnsonii UBLJ-01.

In one embodiment, the composition comprises 0.5 billion cfu of Lactobacillus crispatus UBLCp01, 0.25 billion cfu of Lactobacillus gasseri UBLG36, and 0.25 billion cfu of Lactobacillus johnsonii UBLJ01.

In one embodiment, the composition disclosed herein further comprises an antifungal agent.

In one embodiment, the composition is in the form of a freeze-dried preparation, cream, paste, gel liquid, suppository, capsule, or tablet for vaginal application.

In one embodiment, the composition disclosed herein is in form of vaginal suppository.

One embodiment of the current invention is a method of treating or preventing vaginitis in female subjects, the method comprising the steps of:
(a) providing a composition comprising the following ingredients per single dosage:
(i) a prebiotic: and (ii) dried live bacteria of the strains Lactobacillus crispatus UBLcP-01, Lactobacillus gasseri UBLG-36, and Lactobacillus johnsonii UBLJ-01; and
(b) administering the composition intravaginally to the subject in need of treatment.

In one embodiment, the method disclosed herein, wherein the prebiotic is selected from the group consisting of inulin, Fructooligosaccharides (FOS), Galacto-oligosaccharides (GOS), and lactulose. In one embodiment, the prebiotic is present in the composition at a concentration 25-30% w/w.

In one embodiment, the probiotic composition described herein further comprises, at least one prebiotic, wherein optionally the prebiotic comprises an inulin, Fructooligosaccharides (FOS), Galacto-oligosaccharides (GOS), lactulose, extracts of artichoke, chicory root, oats, barley, various legumes, garlic, kale, beans or flacks or an herb.

In one embodiment, the method disclosed herein, wherein the composition further comprises an antioxidant selected from the group consisting of ascorbic acid, vitamin E, glutathione, coenzyme Q10, beta-carotene, lycopene, vitamin A or derivatives thereof and the antioxidant is present at a concentration of 8-12% w/w in the composition. In one embodiment, the antioxidant is ascorbic acid or a salt thereof. In one embodiment, the ascorbic acid or its salt thereof is present at a concentration of 10% (w/w) in the composition.

In one embodiment, the method disclosed herein, the composition disclosed herein comprises 0.1-5 billion cfu of Lactobacillus crispatus UBLcP-01, 0.1-5 billion cfu of Lactobacillus gasseri UBLG-36, and 0.1-5 billion cfu of Lactobacillus johnsonii UBLJ-01. In one embodiment, the composition disclosed herein comprises 0.1-1 billion cfu of Lactobacillus crispatus UBLcP-01, 0.1-1 billion cfu of Lactobacillus gasseri UBLG-36, and 0.1-1 billion cfu of Lactobacillus johnsonii UBLJ-01. In one embodiment, the composition comprises total 1-3 billion cfu of Lactobacillus crispatus UBLcP-01, Lactobacillus gasseri UBLG-36, and Lactobacillus johnsonii UBLJ-01. In one embodiment, the composition comprises total 1 billion cfu of Lactobacillus crispatus UBLcP-01, Lactobacillus gasseri UBLG-36, and Lactobacillus johnsonii UBLJ-01.

In one embodiment, the method disclosed herein, wherein the composition comprises 0.5 billion cfu of Lactobacillus crispatus UBLCp01, 0.25 billion cfu of Lactobacillus gasseri UBLG36, and 0.25 billion cfu of Lactobacillus johnsonii UBLJ01.

In one embodiment, the method disclosed herein, wherein the composition is in the form of a freeze-dried preparation, cream, paste, gel liquid, suppository, capsule, or tablet for vaginal application.

In one embodiment, the composition is administered to the subject once or twice a day.

In one embodiment, the probiotic bacterial cells in the composition are dried by lyophilization or by spray-drying.

In one embodiment, the method of treating vaginitis by administering the composition disclosed herein, is accompanied by at least one more standard care of treatment for the patient. For example, the patient may undergo any standard care of treatment for BV, or candidiasis or inflammatory symptoms, besides treatment with the composition disclosed herein.

In one embodiment, the composition comprising the three strains and the prebiotic exhibits synergistic function for treating vaginitis. Though each strain disclosed herein exhibits favorable properties such as biofilm production, autoaggregation, coaggregation, EPS production, adhesion to cells, and survival in low pH; but they exhibit varying extents of these favorable characteristics. For example, the composition exhibits much higher co-aggregation with E. coli than any of the individual strains. Also, for example, Lactobacillus gasseri ULG-36 shows good biofilm formation capacity but does not produce H₂O₂ (example 6). Since all these properties are collectively important for reducing infection, inflammation and maintain pH after colonizing vagina by biofilm formation, these three strains collectively have the potential to form biofilms and produce the metabolites important for reducing or treating vaginitis.

In one embodiment, the current invention encompasses methods for treating, ameliorating and/or preventing vaginal infections and inflammatory conditions, including vaginitis using the formulation described herein. The current invention also encompasses methods for making and using this formulation, and also encompasses compositions comprising this formulation and carriers, additives, synbiotics or any other components required for administration of this formulation.

In one embodiment, the probiotic formulation is used to prevent/treat vaginal dysbiosis and maintain a healthy vaginal eco-system. The dried Lactobacillus cells as used herein are viable and are capable of growing in the vaginal system (e.g., the vaginal cavity) of the subject upon administration.

In one embodiment, the invention also encompasses a method of treating or ameliorating an inflammatory condition in a female subject by intravaginally or transvaginally administering to the female subject the composition described herein.

In one embodiment, the current invention encompasses probiotic vaginal formulations, which are useful for treating, preventing or reducing one or more symptoms of a vaginal infection or inflammatory condition or alleviating vaginal infections or inflammatory conditions. In one embodiment, the current invention also encompasses probiotic formulation that can be administered orally, which are useful for treating, preventing or reducing one or more symptoms of a vaginal infection or inflammatory condition or alleviating vaginal infections or inflammatory conditions.

In one embodiment, the probiotic vaginal formulations described herein may relieve symptoms of vaginitis, such as internal and external vaginal burning, itching, and irritation, and may reduce vaginal foul odor.

In one embodiment, the formulation comprises wherein the formulation comprises 0.1-5 billion cfu of Lact. crispatus UBLcP-01, 0.1-1 billion cfu of Lact. johnsonii UBLJ-01, and 0.1-5 billion cfu of Lact. gasseri UBLG-36.

In one embodiment, the composition disclosed herein comprises 0.25 to 1 billion cfu of the Lactobacilli strains disclosed herein.

In one embodiment, the Lactobacillus strains described in the current invention exhibit low pH tolerance, survival in simulated vaginal fluid, adhesion abilities, safety, and antimicrobial potential, exopolysaccharide production, and biofilm-forming abilities.

In one embodiment, the formulation encompassed herein has antimicrobial activity against pathogenic microbes. Examples of such pathogenic microorganisms include, but are not limited to, Escherichia coli, Gardnerella vaginalis, Proteus mirabilis, and Candida albicans.

In one embodiment, the composition disclosed herein further comprises active ingredients such as hormones and/or anti-inflammatory agents and/or bactericidal agents and/or antifungal agents.

In one embodiment, probiotic formulation disclosed herein is used to maintain a beneficial vaginal flora in a woman without an infection. In one embodiment, the formulation is used to restore a beneficial vaginal flora following an antibiotic treatment. The invention thus relates to a pharmaceutical composition comprising one or more of the strains disclosed herein, for use in the maintenance or restoration of a beneficial vaginal flora.

In one embodiment, the current invention encompasses a method of reducing inflammation in vagina of female patients; the method comprising the steps of: administering the composition disclosed herein intravaginally to the subject in need of treatment.

In one embodiment, the composition reduces vaginal inflammation by reducing levels of Il-6, IL-8 and TNF-α in the female patients.

In one embodiment, the composition described herein inhibits growth of Escherichia coli, Gardnerella vaginalis, Proteus mirabilis, and Candida albicans in the vagina of the female patients.

In one embodiment, the bacteria cells in the composition described herein exhibit low pH tolerance, good survival in simulated vaginal fluid, adhesion to vaginal epithelial cells, and antimicrobial properties, exopolysaccharide production.

In one embodiment the composition described herein exhibits biofilm-forming abilities.

In one embodiment, the combination of these three strains exhibits synergistic properties compared to the individual bacterial strains Lactobacillus crispatus UBLcP-01, Lactobacillus gasseri UBLG-36, and Lactobacillus johnsonii UBLJ-01.

In one embodiment, the composition described herein further comprises an anti-microbial agent or preparation. Examples of an anti-microbial agent include, but are not limited to, recombinant proteins e.g. human soluble serine leukocyte protease inhibitor (SLPI), which is significantly reduced by BV bacteria and Trichomonas vaginalis and deficient in the vaginal secretions of women with BV and trichomoniasis (Huppert et al., 2013), synthetic small molecules e.g. purine analogs e.g. 9-(2-deoxy-2-fluoro-β-Darabinofuranosyl) adenine.

In one embodiment, the antimicrobial agent is an antibiotic. In one embodiment, the antibiotic is metronidazole. In another one embodiment, the antibiotic is clindamycin.

### EXAMPLES

Bacterial Strains

Lactobacillus crispatus UBLcP-01, Lactobacillus gasseri UBLG-36, and Lactobacillus johnsonii UBLJ-01 isolated from the vagina of healthy reproductive age Indian women were screened for beneficial probiotic properties. These strains showed the ability to survive acidic and simulated vaginal fluid conditions and could adhere to mucin. Lactobacillus gasseri UBLG-36, and Lactobacillus johnsoni UBLJ-01 produced d- and l-lactic acid, whereas Lactobacillus crispatus UBLcP-0 produced hydrogen peroxide and d- and l-lactic acid. All strains inhibited the growth of pathogens (Escherichia coli, Gardnerella vaginalis, Proteus mirabilis, and Candida albicans) and were capable of co-aggregating with them with varying degrees.

### Example 1: Survival Under In Vitro Vaginal Conditions

### Example 1.1: Growth at Low pH

One-milliliter overnight-grown bacterial cultures obtained from single colony inoculation in 10 mL MRS broth (HiMedia, Mumbai, India) were each inoculated separately in 100 mL MRS broth adjusted to pH 3.5, 4.0, and 4.5 using lactic acid (Sisco Research Laboratories, Mumbai, India). All the cultures were incubated anaerobically at 37° C. for up to 48 h. The growth was determined on MRS agar plates at 0, 24, and 48 h and expressed in log₁₀ CFU/mL. Simultaneously, the appropriate sample dilutions were stained with LIVE/DEAD™ BacLight™ bacterial viability kit (Thermo Scientific, Massachusetts, USA) and analyzed on CytoFLEX flow cytometer (Beckman Coulter, Indianapolis, USA). The analysis was conducted at a slow flow rate (10 μL/min) and viability was expressed as mean log₁₀ CFU/mL. The bacterial growth in MRS broth (pH 6.5) served as a control.

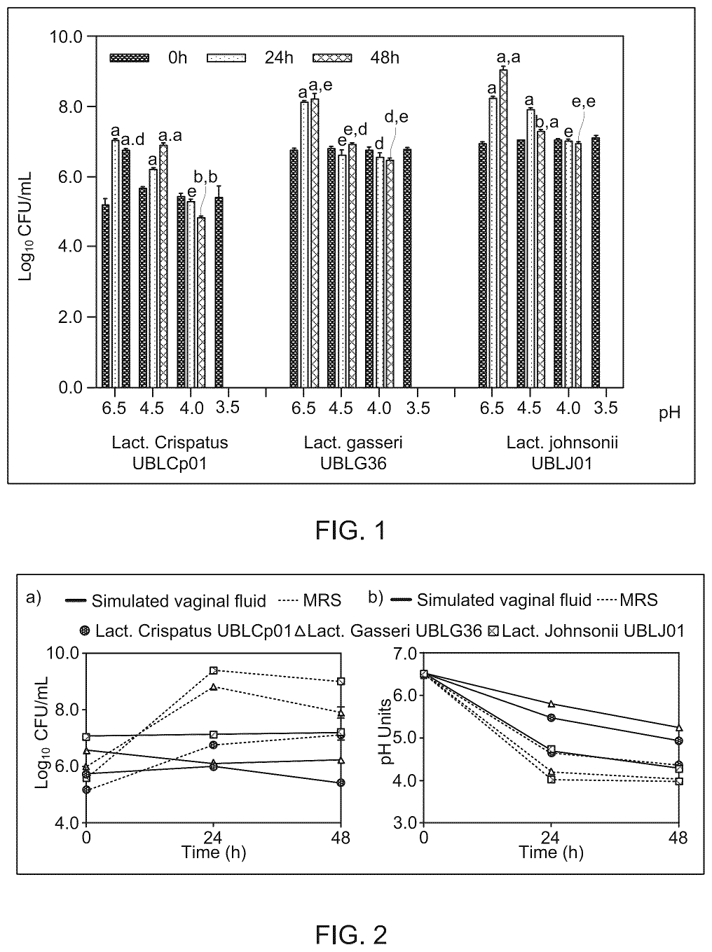

The survival of Lact. crispatus UBLcP-01, and Lact. gasseri UBLG-36 was found to be significantly reduced to 89% and 95% when cultivated in acidified MRS (pH 4.0; adjusted with lactic acid, a representative acid from the vaginal tract) after 48 h. Simultaneously, no significant changes in growth (98%) were noticed when Lact. johnsonii UBLJ-01 was cultivated in acidified MRS (pH 4.0) after 48 h (FIG. 1). FIG. 1 shows bacterial growth in low pH (6.5, 4.5, 4.0, and 3.5) media (acidified with lactic acid) at 0, 24, and 48 h of anaerobic incubation. All data are represented as mean±SD. a, p≤0.0001; b, p≤0.001; d, p≤0.05; and e, p>0.05.

Besides this, at pH 4.5 and 6.5, all the cultures showed more than 100% growth at 48 h. At pH 3.5, no bacterial survival was detected by using the traditional plate count method (FIG. 1).

### Example 1.2: Growth in Simulated Vaginal Fluid

One-milliliter overnight-grown bacterial cultures were each inoculated separately in 100 mL simulated vaginal fluid (SVF) prepared as per the composition given in Ahire et al (Ref 1) and herein, consisting part I (g/L) (all reagents were obtained from Sigma, HiMedia, Mumbai, India or Sisco Research Laboratories, Mumbai, India): NaCl, 3.5; KCl, 1.5; K₂HPO₄, 1.74; K H₂PO₄, 1.36; dextrose, 10.8, cysteine HCl, 0.5; part II (%): glycogen, 0.1; mucin (purified), 0.025; Tween 20, 0.02; urea, 0.05: vitamin K1, 0.01: hemin, 0.05: albumin, 0.2; MgSO₄, 0.03; NaHCO₃, 0.004; part III (mg/mL): biotin, 0.005: myo-inositol, 50.0; niacinamide, 0.5: pyridoxine HCl, 0.5; thiamine HCl, 0.5; d-calcium pantothenate, 0.5; folic acid, 0.5; p-aminobenzoic acid, 0.01: choline chloride, 0.5; riboflavin, 0.1: l-ascorbic acid, 1.0; vitamin A (retinol), 0.005; vitamin D (cholecalciferol), 0.005; vitamin B₁₂, 0.01] and incubated anaerobically at 37° C. for up to 48 h. Bacterial growth and pH were determined at 0, 24, and 48 h time intervals. The survivability was determined on MRS agar plates and expressed in log₁₀ CFU/mL. Bacterial growth in MRS served as a control.

After 48 h, both Lact. crispatus UBLcP-01, and Lact. gasseri UBLG-36 showed 94% survival in SVF, whereas Lact. johnsonii UBLJ-01 showed 102% survival (FIG. 2a). The changes in pH of SVF recorded after 48 h of incubation were decreased from 6.52±0.01 to 4.93±0.005 (UBLcP-01), 5.25±0.01 (UBLG-36), and 4.28±0.005 (UBLJ-01), respectively (FIG. 2b). In MRS (pH 6.50±0.1), UBLcP-01, UBLG-36, and UBLJ-01 showed significantly higher growth (137%; 131%; 161%) and good ability to lower pH (4.36±0.005; 4.01±0.005; 3.97±0.00 units) at 48 h incubation (FIG. 2a, b).

### Example 2: Adhesion Abilities

### Example 2. 1: Adhesion to Mucin

The bacterial adhesion to mucin was performed as described by Ahire et al. [Ref. 1] with certain modifications. In brief, the mucin purchased from Sigma-Aldrich (Sigma-Aldrich, St. Louis, Missouri, USA) was purified further and used for coating. A 2 mL purified mucin solution (100 μg/mL) prepared in 0.05 M Na₂CO₃ (pH 9.7) was dispensed in each well of a 6-well tissue culture plate (Thermo Scientific) and incubated overnight at 4° C. with slow rotations to facilitate mucin coating. After incubation, the solution in wells was discarded and wells were filled with 2 mL phosphate buffer saline (PBS, pH 7.3) containing 1% (w/v) Tween 20. The well-plate was incubated for an hour at 28° C. without rotation. After that, the wells were carefully washed with PBS containing 0.05% (w/v) Tween 20 and inoculated with 2 mL respective culture suspension (0.5 O D_{600 nm}) prepared in PBS containing 0.05% (w/v) Tween 20 (pH 7.3). All the plates were incubated anaerobically at 4° C. for 24 h. After incubation, the bacterial suspension was carefully decanted and wells were gently washed with PBS (Tween 20, 0.05% w/v) and air-dried. The bacteria attached to the mucin-coated wells were stained with 0.1% (v/v) crystal violet and visualized under Olympus inverted microscope (CKX53, Tokyo. Japan). The quantification of bacterial adhesion was performed as per the method described by Ahire (Ref 1).

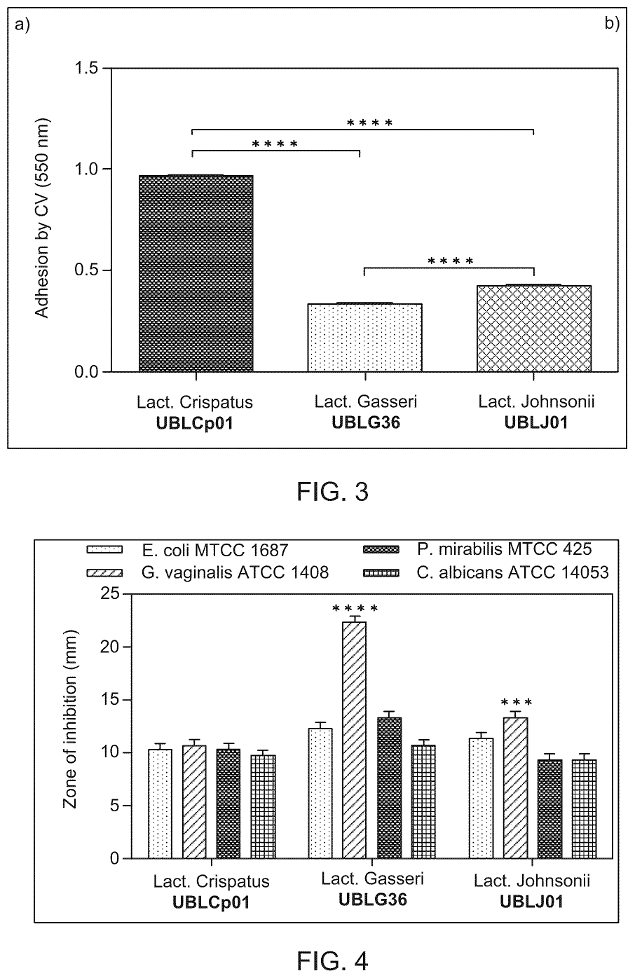

Lact. crispatus UBLcP-01 showed significantly higher (OD₅₅₀=0.97±0.005) adhesion to mucin compared with Lact. johnsonii UBLJ-01 (OD₅₅₀=0.42±0.01) and Lact. gasseri UBLG-36 (OD₅₅₀=0.33±0.002) (FIG. 3).

### Example 3: Zeta Potential

Overnight-grown bacterial cultures were each harvested, washed twice with PBS (pH 7.3), and resuspended in the same to get 0.5 OD units at 600 nm. The DTS1070 capillary cell was filled with bacterial suspension and zeta potential was measured by using Nano-ZS Zetasizer (Malvern, UK) as described by Ahire et al. [Ref 1].

Lact. gasseri UBLG-36 cells showed significantly higher (−11.6±0.35 mV) zeta potential compared to Lact. johnsonii UBLJ-01 (−10.2±0.75 mV) and Lact. crispatus UBLcP-01 (−7.2±0.05 mV) (Table 2).

### Example 4: Aggregation Properties

### Example 4.1: Auto-Aggregation

Auto-aggregation was determined as per the method described herein and in Ahire et al (Ref 1). In brief, bacteria were grown anaerobically at 37° C. for 18 h in MRS broth. The cells were harvested by centrifugation at 11,000×g for 10 min at 4° C. and washed twice with PBS (pH 7.3). The pellets were dissolved in PBS (pH 7.3) to 0.5 OD units at 600 nm (a). A 4 mL bacterial suspension was mixed uniformly for 10 s and incubated anaerobically at 37° C. for 1 h. After incubation, 2 mL upper suspension was removed carefully and absorbance was measured at 600 nm (b). Auto-aggregation percentages were expressed as, (a600 nm−b600 nm/a600 nm)×100.

Lact. gasseri UBLG-36 showed significantly higher auto-aggregation (32.98±1.67%) compared to Lact. crispatus UBLcP-01 (16.19±1.28%) after 1 h. However, the auto-aggregation differences recorded between UBLG-36 (32.98±1.67%) and Lact. johnsonii UBLJ-01 (30.14±1.41%) were insignificant (Table 2).

### Example 4.2: Co-aggregation

Probiotic and pathogen (Escherichia coli MTCC 1687, Gardnerella vaginalis ATCC 14018, Proteus mirabilis MTCC 425, and Candida albicans ATCC 14053) were cultivated overnight at 37° C. in respective growth media and conditions. Culture suspension of 0.5 OD units was prepared as per the method described earlier. The co-aggregation was determined as given herein. 2 mL aliquots of probiotic and pathogen suspension were gently vortexed for 10 s by using Spinix vortex (Tarsons, Kolkata, India) and incubated anaerobically at 37° C. for 1 h. Aliquots (4 mL) of a single bacterial suspension (probiotic or pathogen) served as a control. After incubation, the upper suspension was removed carefully and absorbance was measured at 600 nm. Co-aggregation percentages were expressed as, ((ODx+ODy)/2)−OD (x+y)/ODx+ODy/2×100.

ODx and ODy are the individual aggregation optical densities of the probiotic and the pathogen, and OD(x+y) is the combined aggregation optical density of the probiotic and the pathogen.

All three strains showed co-aggregation, but to different extents. Lact. crispatus UBLcP-01 showed significantly higher coaggregation ability toward E. coli (7.93±0.13%). G. vaginalis (33.11±0.31%), Pr. mirabilis (16.58±0.17%), C. albicans (19.35±0.23%) compared with Lact. gasseri UBLG-36 and Lact. johnsonii UBLJ-01 at 1 h (Table 2). The co-aggregation results of UBLG-36 and UBLJ-01 are presented in Table 2.

### Example 5: Antimicrobial Potential

### Example 5.1: Antimicrobial Activity

Overnight-grown bacterial cultures were spot inoculated (5 μL) onto the dry surface of MRS agar plates and incubated anaerobically at 37° C. for 24-48 h. Simultaneously. E. coli MTCC 1687, G. vaginalis ATCC 14018, Pr. mirabilis MTCC 425, and C. albicans ATCC 14053 were cultivated as per their optimal growth conditions. ˜10⁶ CFU of each pathogen were inoculated separately in 20 mL molten Mueller-Hinton agar and poured on top of the probiotic growth. All the plates were incubated at 37° C. for 24 h and the zone of clearance surrounding the probiotic growth was measured in mm.

After incubation, the culture spots were developed to the average size of 6.66±0.65 mm and showed the inhibition of the selected pathogen. The highest zone of inhibition was recorded against G. vaginalis by Lact. gasseri UBLG-36 (22.33±0.57 mm), whereas the least was against Pr. mirabilis, and C. albicans by Lact. johnsonii UBLJ-01 (9.33±0.61; 9.33±0.57 mm) (FIG. 4). Lact. crispatus UBLcP-01 showed pathogen inhibition zones in the range of 9.60 to 10.60 mm, which are statistically insignificant (FIG. 4).

### Example 5.2: H₂O Production

Hydrogen peroxide (H₂O₂) production was evaluated as given in Ahire et al (Ref 1). Overnight grown bacterial cultures were streaked on MRS agar containing 1 mM 3,3′,5,5′-tetramethyl-benzidine [(SigmaAldrich), prepared in methanol] and 2 U/mL peroxidase (Sigma-Aldrich), and incubated anaerobically at 37° C. for 48 h. After incubation, plates were exposed to air and H₂O₂ production was scored based on the time required to form blue coloration. The results were scored as 0 (no coloration), 1 (low, >20 min), 2 (medium, 10-20 min), and 3 (high, <10 min).

d- and L-Lactic Acid Production

Overnight-grown bacterial cultures were each inoculated (1%, v/v) separately in MRS broth and incubated anaerobically at 37° C. for 24 h. Supernatants were obtained by centrifugation at 11,000×g for 10 min (4° C.) and concentrations of d- and l-lactic acid were measured using an NZY Tech, d-/l-lactic acid kit (Lisboa, Portugal), according to the manufacturer's instructions.

Lact. crispatus UBLcP-01 showed intense blue color formation in less than 10 min after air exposure, indicating high (scored 3) hydrogen peroxide production by the strain. Besides this, Lact. gasseri UBLG-36 and Lact. johnsonii UBLJ-01 failed to produce any color (Scored 0). After 24 h of anaerobic incubation, UBLcP-01, UBLG-36, and UBLJ-01 produced 26.08, 23.64, and 46.90 mmol/L of d-lactic acid and 34.08, 33.74, and 59.28 mmol/L l-lactic acid, respectively.

### Example 5.3: Exopolysaccharide Production and Quantification

Exopolysaccharide (EPS) production was performed as described. In brief, overnight-grown bacterial cultures were spot inoculated (5 μL) on ruthenium red milk agar [consisting (g/L): yeast extract, 5; skim milk powder, 100: sucrose, 10; agar, 15: ruthenium red, 0.08 (obtained from Sigma chemicals or Himedia) and incubated anaerobically at 37° C. for 48 h. After incubation, the plates were observed for white (ropy/EPS producer) and/or red (non-ropy/non-producers) growth patterns.

Overnight-grown ropy bacterial strains were each inoculated (1%, v/v) separately in MRS broth and incubated anaerobically at 37° C. for 48 h. The cell-free supernatant obtained by centrifugation (11,000×g for 10 min, 4° C.) was mixed with double volume ice-cold (−80° C.) absolute ethanol and kept at 4° C. for 48 h. The precipitated EPS was collected by centrifugation (5000×g, 10 min, 4° C.) and dissolved in 8% (w/v) trichloroacetic acid (TCA) and kept overnight at 4° C. The precipitated proteins were discarded by centrifugation (5000×g, 10 min, 4° C.) and the supernatant was mixed with double volume ice-cold (−80° C.) absolute ethanol. The mixture was kept at 4° C. for 48 h to precipitate EPS. The process of ethanol and TCA treatment was repeated twice. The collected EPS was dialysed against ultra-pure water at 4° C. for 48 h, using a 10 kDa cut-off dialysis membrane (HiMedia). During the dialysis, ultra-pure water was replaced at 12 h intervals. The dialysed EPS was lyophilised (Lyo lab. United States), weight was measured and stored at 4° C.

In another experiment, EPS was evaluated for total carbohydrate by anthrone reagent method and protein by Pierce™ bicinchoninic acid (BCA) kit (Thermo Fisher Scientific, United States), as per manufacturer's instructions. Adhesion to xylene shows the hydrophobicity of the bacterial cells, which in turn is an indication of the ability of the cells to adhere to epithelial cells.

After 48 h incubation, all the strains showed white growth on ruthenium red milk agar plates, which was indicative of EPS production. Lact. johnsonii UBLJ-01 produced the highest amount of EPS (144.0 mg/L) compared to Lact. gasseri UBLG-36 (130.0 mg/L), and Lact. crispatus UBLcP-01 (120.0 mg/L). The total carbohydrate of EPS produced by strains was 422.5±10.9 μg (UBLcP-01), 472.9±8.6 μg (UBLG-36), and 504.4±10.5 μg (UBLJ-01) per mg of EPS. The protein composition was 2.88±1.74 μg (UBLcP-01), 9.83±1.66 μg (UBLG-36), and 7.05±3.15 μg (UBLJ-01) per mg of EPS.

### Example 6: Biofilm Formation

Overnight-grown bacterial cultures were diluted separately in MRS broth and 200 μL quantities were dispensed into 96-well plates (Nunclon™ Delta surface, Thermo Scientific). The plates were incubated anaerobically at 37° C. for 24 h. Total biofilm formation, viable cells in biofilm, and planktonic cells were estimated at 0- and 24-h time intervals as described by Ahire (Ref 1). Experiments were performed in six replicates.

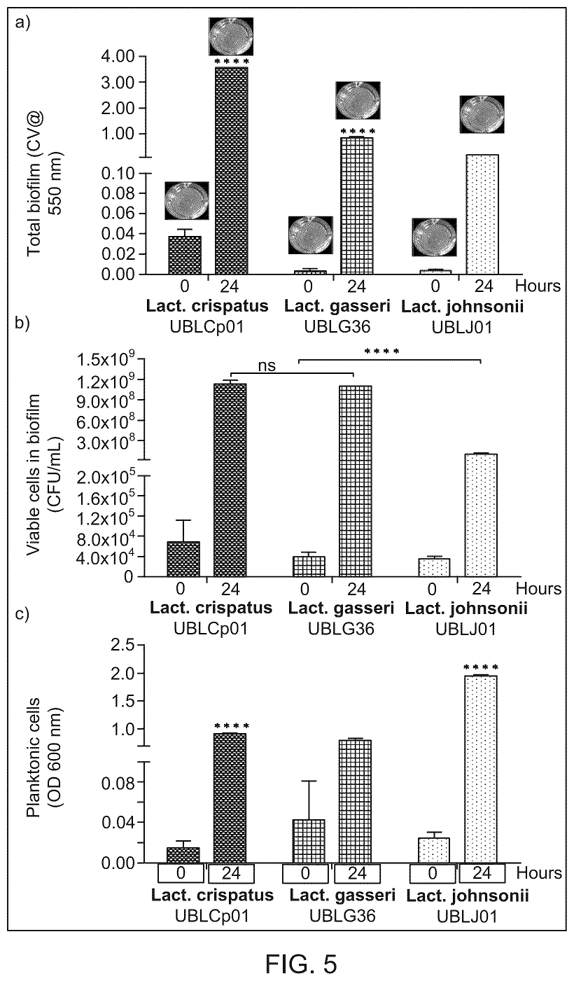

After 24 h, the total biofilm formation estimated by crystal violet staining showed significantly higher OD readings for Lact. crispatus UBLcP-01 (3.54±0.02) than Lact. gasseri UBLG-36 (0.82±0.06), and Lact. johnsoni UBLJ-01 (0.17±0.04) (FIG. 5a). The viable cells in biofilm of UBLcP-01 (1.13×10⁹) and UBLG-36 (1.10×10⁹) were significantly higher compared to UBLJ-01 (9.0×10⁷) (FIG. 5b).

Furthermore, the plank-tonic (free) cell OD readings recorded for UBLJ-01 (1.95±0.02) were significantly higher compared to UBLcP-01 (0.90±0.02), and UBLG-36 (0.80±0.03). The differences recorded between UBLcP-01 and UBLG-36 were significant (FIG. 5c).

TABLE 2
In vitro adhesion to xylene, zeta potential, autoaggregation and co-aggregation abilities of Lactobacillus strains

| | | | | Co-aggregation (%) ^{#} | | | |
|---|---|---|---|---|---|---|---|
| | Adhesion | Zeta | | Escherichia | Gardnerella | Proteus | Candida |
| Lactobacillus | to Xylene | potential | Autoaggregation | coli | vaginalis | mirabilis | albicans |
| species | (%) ^{#} | (mV) | (%) ^{#} | MTCC 1687 | ATCC 14018 | MTCC 425 | ATCC 14053 |
| Lact. | 48.05 ± | −7.2 ± | 16.19 ± | 7.93 ± | 33.11 ± | 16.58 ± | 19.35 ± |
| crispatus | 6.91d | 0.05 | 1.28 | 0.13a | 0.31a | 0.17a | 0.23a |
| UBLCP-01 | | | | | | | |
| Lact. | 34.98 ± | −11.6 ± | 32.98 ± | 1.15 ± | 18.45 ± | 5.02 ± | 7.22 ± |
| gasseri | 2.86 | 0.35 a, d | 1.67 a, e | 0.10 | 0.21a | 0.10 | 0.18b |
| UBLG-36 | | | | | | | |
| Lact. | 32.32 ± | −10.2 ± | 30.14 ± | 2.90 ± | 9.32 ± | 5.65 ± | 6.03 ± |
| johnsonii | 3.98 | 0.75 | 1.41 | 0.12a | 0.15 | 0.21c | 0.10 |
| UBLJ-01 | | | | | | | |

(^{#} values for one hour;
ap ≤ 0.0001;
bp ≤ 0.001;
cp ≤ 0.01;
dp ≤ 0.05;
e: p > 0.05, not significant)

Statistical Analysis

All the experiments were performed in three replicates or else stated in methods. The data are expressed as a mean±standard deviation (SD). GraphPad Prism (San Diego, California, USA) was used to perform significant differences between means by Tukey's test after analysis of variance (ANOVA). The p value<0.05 was Considered Statistically Significant.

### Example 6: Synergistic Properties of the Blend of the Three Probiotic Strains Combined Together

The cumulative effect of the three strains was studied to test whether single culture or in combination will be a better choice for the treatment of bacterial vaginosis. Hence, the strength of the formula was fixed to a total of 1 billion of viable cultures comprising of Lactobacillus crispatus UBLcP-01, Lactobacillus gasseri UBLG-36, and Lactobacillus johnsonii UBLJ-01 at different strengths.

TABLE 3
number of bacterial cells of the three individual strains in the three
different blends (mixtures) tested

| Formulation | L crispatus | L gasseri | L johnsoni | Total strength |
|---|---|---|---|---|
| B1 | 0.5 bn | 0.25 bn | 0.25 bn | 1 bn |
| B2 | 0.25 bn | 0.5 bn | 0.25 bn | 1 bn |
| B3 | 0.25 bn | 0.25 bn | 0.5 bn | 1 bn |
| 4 (Lc only) | 1.0 bn | 0 | 0 | 1 bn |
| 5 (Lg only) | 0 | 1.0 bn | 0 | 1 bn |
| 6 (Lj only) | 0 | 0 | 1.0 bn | 1 bn |

Preparation of the blend of three bacterial strains Lactobacillus crispatus UBLcP-01, Lactobacillus gasseri UBLG-36, and Lactobacillus johnsonii UBLJ-01 for autoaggregation, co aggregation, Zeta potential and biofilm formation is shown in Table 3.

The blend powder equivalent to 1 gm was weighed and suspended in to 3 ml of PBS and incubated for 2 h to allow the proper suspension of the culture. Then the OD was adjusted to 0.5 and used for various experiments as described below.

The studies were done as described in the previous examples 1-5.

### Example 6.1: Autoaggregation and Coaggregation of the Compositions/Blends of the Three Strains Lactobacillus crispatus UBLcP-01, Lactobacillus gasseri UBLG-36, and Lactobacillus johnsonii UBLJ-01

The autoaggregation helps in bacteria protecting itself from unsuitable environmental conditions. The observations suggest that the percentage of autoaggregation follows the order B2>B3>B1 as shown in Table 3.

The coaggregation for B1 showed antimicrobial activity in the order G vaginalis>P mirabilis>E. coli>C albicans, for B2 it follows the order C albicans>G vaginalis>P mirabilis>E. coli and for Blend 3 it follows the order C albicans>E. coli>G vaginalis>P mirabilis. The data suggest that blend 1 has the comparatively more potential to coaggregate G vagina/is compared to other blends.

TABLE 4
Studies on Auto-aggregation and Co-aggregation

| | Auto aggregation | Co-aggregation (%) | | | |
|---|---|---|---|---|---|
| Samples | (%) | E. coli | C albicans | G vaginalis | P mirabilis |
| B1 | 27.98 ± 1.51 | 11.08 ± 1.92 | 5.07 ± 0.92 | 28.45 ± 1.89 | 13.1 ± 2.34 |
| B2 | 40.8 ± 1.71 | 11.05 ± 0.69 | 34.19 ± 0.70 | 15.675 ± 0.95 | 12.89 ± 1.24 |
| B3 | 38.02 ± 0.83 | 15.31 ± 1.05 | 34.62 ± 2.30 | 10.345 ± 0.77 | 7.97 ± 1.42 |
| Lc | 16.01 ± 1.52 | 6.89 ± 0.78 | 18.95 ± 0.57 | 30.05 ± 1.62 | 16 ± 1.52 |
| Lj | 34.023 ± 1.17 | 2.98 ± 0.63 | 8.12 ± 0.15 | 15.65 ± 0.82 | 5 ± 1.70 |
| Lg | 36.035 ± 1.43 | 1.20 ± 0.20 | 12.34 ± 2.83 | 7.65 ± 0.62 | 5 ± 0.60 |

### Example 6.2: Biofilm Formation of the Compositions/Blends of the Three Strains Lactobacillus crispatus UBLcP-01, Lactobacillus gasseri UBLG-36, and Lactobacillus johnsonii UBLJ-01

The ability to form biofilm helps in preventing the adherence of pathogenic bacteria to the cells. Thus protects the host from the pathogens. It was observed that the potential to form biofilm is comparatively more for B1 followed by B2 and B3. Thus, viable cells were 30% more in biofilm of B1 than when compared to B3, this variation is in significant when compared with B2. The number of planktonic cells exhibited significant variation between B1 and B3. However, the variation is insignificant between B1 and B2. Table 5 shows the comparative data for biofilm formation.

TABLE 5
Biofilm formation potential

| | | | | OD of |
|---|---|---|---|---|
| | IC | FC | Crystal violet | planktonic |
| Samples | (initial count)* | (final count)** | (OD) | cells |
| B1 | 1.7*106 ± 0.64 | 9.16*108 ± 0.64 | 1.972 ± 0.154 | 0.112 ± 0.01 |
| B2 | 1.35*106 ± 0.52 | 8.34*108 ± 0.59 | 1.796 ± 0.32 | 0.135 ± 0.02 |
| B3 | 1.69*106 ± 0.64 | 6.72*108 ± 0.70 | 1.34 ± 0.31 | 0.231 ± 0.01 |
| Lc | 2*106 ± 0.65 | 9.43*108 ± 0.80 | 1.878 ± 0.22 | 0.109 ± 0.00 |
| Lj | 2*106 ± 0.66 | 2*106 ± 0.36 | 0.356 ± 0.13 | 0.926 ± 0.14 |
| Lg | 4*106 ± 1.49 | 8.32*108 ± 0.55 | 1.923 ± 0.13 | 0.123 ± 0.02 |

IC refers to number of cells seeded in to the plate
FC refers to number of cells after incubating the plate for 24 h

TABLE 6
Studies on Antimicrobial potential

| | | Antimicrobial activity | | | |
|---|---|---|---|---|---|
| Samples | Zeta potential | E. coli | C albicans | G vaginalis | P mirabilis |
| B1 | −13.5 ± 0.99 | 8 ± 0.82 | 9 ± 0.82 | 21 ± 1.63 | 13 ± 1.63 |
| B2 | −12.3 ± 1.52 | 11 ± 1.41 | 14 ± 1.41 | 20 ± 1.63 | 13 ± 0.82 |
| B3 | −14.7 ± 0.92 | 11 ± 1.41 | 9 ± 0.82 | 10 ± 1.41 | 9 ± 0.82 |
| Lc | −8.83 ± 1.15 | 8 ± 0.82 | 9 ± 0.82 | 9 ± 4.24 | 10 ± 0.82 |
| Lj | −12.3 ± 1.74 | 10 ± 0.82 | 7 ± 0.00 | 12 ± 0.82 | 11 ± 0.82 |
| Lg | −16.7 ± 1.51 | 12 ± 2.16 | 13 ± 0.82 | 21 ± 0.82 | 14 ± 0.82 |

The antimicrobial potential followed the order G vaginalis>P mirabilis>C albicans>E. coli, for B2 it follows the order G vaginalis>C albicans>P mirabilis>E. coli and blend 3 do not show significant variation in antimicrobial potential against different pathogens (Table 6).

The zeta potential of bacteria serves as an indicator for their viability and many other properties such as adhesion. The Zeta potential of various blends did not exhibit significant variation when compared to each other.

The potential to produce hydrogen peroxide was observed to be high with B1 when compared to B2 and B3. The reason being that the concentration of L crispatus is more in B1 compared to B2 and B3. Among individual cells, only L crispatus has the potential to produce hydrogen peroxide. Thus it follows the order B1>B2 and B3.

TABLE 7
Production of H₂O₂

| Sample | Score | Distribution of blue colonies |
|---|---|---|
| B1 | 3 | More |
| B2 | 3 | Few |
| B3 | 3 | Few |
| Lc | 3 | NA |
| Lj | 0 | NA |
| Lg | 0 | NA |

### Example 6.3: Adhesion Abilities

Adhesion studies were carried out VK2 cell line obtained ATCC (CRL-2616). The cells were plated in to 12 well plate at 2*10\hat{ }5 cells/ml in Keratinocyte serum free medium (Gibco) and incubated in CO2 incubator till the cells reached confluency. Then the cells were replenished with fresh Keratinocyte serum free medium (KSF) and treated the cells with blend powder containing 10*8 cells. Simultaneously, individual bacterial cells were also used for the treatment of VK2 cells for 2 h. Then the cells were washed thrice with PBS and stained with Geimsa stain followed by enumeration of number of cells adhered in twenty different microscopic fields.

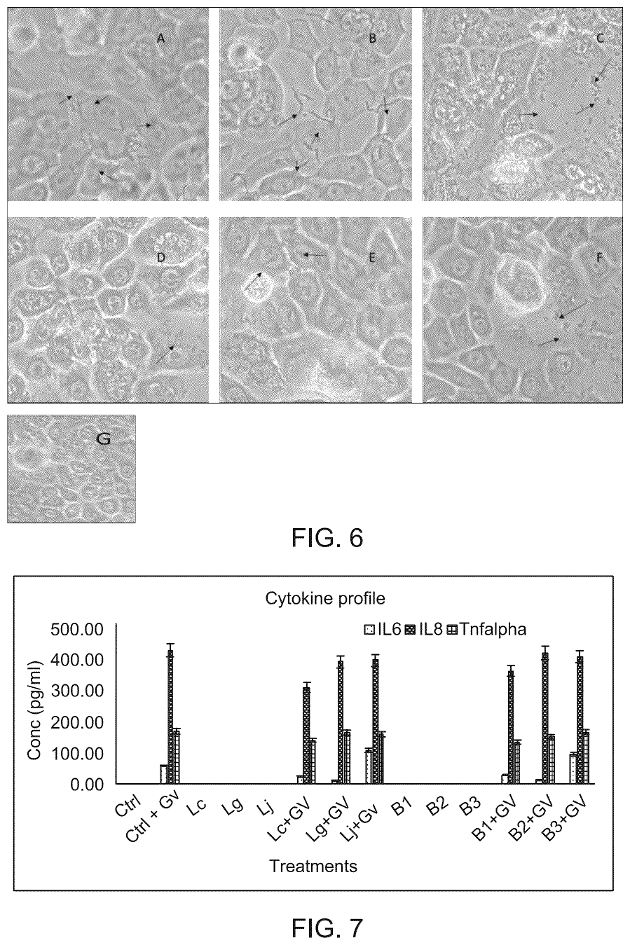

The adhesion studies for all the blends revealed that there are more than 100 bacterial cells attached to VK2 cells when observed in 20 different microscopic fields. Hence the data suggest the bacterial cells in blends exhibited good adhesion potential (FIG. 6).

FIG. 6 shows comparison of Adhesion of individual cells and Blends of the three strains to vaginal epithelial cells (VK2 cells).

The figure shows VK2 cells treated with (A) Lactobacillus crispatus UBLcP-01 (B) Lactobacillus gasseri UBLG-36 (C) Lactobacillus johnsonii UBLJ-01 (D) Blend1, (E) Blend 2, (F) Blend 3 (G) Control

It was observed that in the absence of GV neither blends nor cultures enhance the production of the proinflammatory genes. Further in presence of Gardnerella vaginalis, the cultures were effective in down regulating the production of these cytokines. In presence of Gardnerella vaginalis blend1 (B1) effectively down regulated IL8 and TNFα. However, blend 2 was effective in down regulating IL6. When compared to B1 and B2, B3 has upregulated IL6.

### Example 6.4: Effect of Blends/Compositions of all the Three Strains and the Individual Strains on Anti-Inflammatory Cytokines

In a 24 well plate cells were plated at a concentration of 1*10⁵ and allowed to reach near confluence. Then the cells were replenished with fresh (KSF) medium followed by treatment with Lactobacillus crispatus UBLcP-01, Lactobacillus gasseri UBLG-36 and Lactobacillus johnsonii UBLJ-01 and various blends described in Table 3, in the presence and absence of GV for 24 h. Then supernatants were collected and stored at −80° C. to analyse for the change in levels of IL-4, IL-6, IL-8 and TNFalpha cells.

It was observed that in the absence of GV neither blends nor cultures enhance the production of the proinflammatory genes. Further in presence of Gardnerella vaginalis, the cultures were effective in down regulating the production of these cytokines. In presence of Gardnerella vaginalis, blend1 (B1) effectively down regulated IL8 and TNFalpha. However, blend 2 was effective in down regulating IL6. When compared to B1 and B2, B3 has upregulated IL6.

FIG. 7 shows the cytokine profile in with and without GV in presence of Blend 1.

Hence all three blends showed better overall properties such as biofilm production, antimicrobial potential, coaggregation, compared to the three individual strains. There was some variation in the individual activities shown by the three blends, but overall all three compositions shows good activity. Blend B1 exhibited better properties of Coaggregation, antimicrobial potential, Biofilm formation and the reduction of IL8 when compared to B2 and B3 powders. Furthermore, Blend 1 exhibited good antimicrobial potential against Gardnerella vaginalis when compared to any of individual culture alone.

We claim:

1. A composition for treating vaginitis comprising dried live bacteria of the strains Lactobacillus crispatus UBLCp01, Lactobacillus gasseri UBLG36, Lactobacillus johnsonii UBLJ01 and a prebiotic.

2. The composition of claim 1, wherein the prebiotic is selected from the group consisting of inulin, Fructooligosaccharides (FOS), Galacto-oligosaccharides (GOS), and lactulose.

3. The composition of claim 1, wherein the prebiotic is present in the composition at a concentration 25-30% w/w.

4. The composition of claim 1, wherein it further comprises an antioxidant selected from the group consisting of vitamin C, vitamin E, glutathione, coenzyme Q10, beta-carotene, lycopene, vitamin A or derivatives thereof and the antioxidant is present at a concentration of 8-12% w/w in the composition.

5. The composition of claim 1, wherein the antioxidant is ascorbic acid or a salt thereof.

6. The composition of claim 1, wherein the ascorbic acid or its salt thereof is present at a concentration of 10% (w/w) in the composition.

7. The composition of claim 1, wherein it comprises 0.1-5 billion cfu of Lactobacillus crispatus UBLCp01, 0.1-5 billion cfu of Lactobacillus gasseri UBLG36, and 0.1-5 billion cfu of Lactobacillus johnsonii UBLJ01.

8. The composition of claim 1, wherein it comprises total 1 billion cfu of Lactobacillus crispatus UBLCp01, Lactobacillus gasseri UBLG36, and Lactobacillus johnsonii UBLJ01.

9. The composition of claim 1, wherein it comprises 0.5 billion cfu of Lactobacillus crispatus UBLCp01, 0.25 billion cfu of Lactobacillus gasseri UBLG36, and 0.25 billion cfu of Lactobacillus johnsonii UBLJ01.

10. The composition of claim 1, wherein it further comprises an antifungal agent.

11. The composition of claim 1, wherein the composition is in the form of a freeze-dried preparation, cream, paste, gel liquid, suppository, capsule, or tablet for vaginal application.

12. The composition of claim 11, wherein it is in form of vaginal suppository.

13. A method of treating or preventing vaginitis in female subjects, the method comprising the steps of:
(a) providing a composition comprising the following ingredients per single dosage:
i. a prebiotic; and
ii. dried live bacteria of the strains Lactobacillus crispatus UBLCp01, Lactobacillus gasseri UBLG36, and Lactobacillus johnsonii UBLJ0; and
(b) administering the composition intravaginally to the subject in need of treatment.

14. The method of claim 13, wherein the prebiotic is selected from the group consisting of inulin, Fructooligosaccharides (FOS), Galacto-oligosaccharides (GOS), and lactulose.

15. The method of claim 13, wherein the prebiotic is present in the composition at a concentration 25-30% w/w.

16. The method of claim 13, wherein it further comprises an antioxidant selected from the group consisting of ascorbic acid, vitamin E, glutathione, coenzyme Q10, beta-carotene, lycopene, vitamin A or derivatives thereof and the antioxidant is present at a concentration of 8-12% w/w in the composition.

17. The method of claim 16, wherein the antioxidant is ascorbic acid or a salt thereof and it is present at a concentration of 10% (w/w) in the composition.

18. The method of claim 13 wherein the composition comprises 0.1-5 billion cfu of Lactobacillus crispatus UBLCp01, 0.1-5 billion cfu of Lactobacillus gasseri UBLG36, and 0.1-5 billion cfu of Lactobacillus johnsonii UBLJ01.

19. The method of claim 13, wherein the composition comprises 0.5 billion cfu of Lactobacillus crispatus UBLCp01, 0.25 billion cfu of Lactobacillus gasseri UBLG36, and 0.25 billion cfu of Lactobacillus johnsonii UBLJ01.

20. The method of claim 13, wherein the composition is in the form of a freeze-dried preparation, cream, paste, gel liquid, suppository, capsule, or tablet for vaginal application.

21. The method of claim 13, wherein the composition is administered to the subject once or twice a day.
